(19) United States
(12) Patent Application Publication
(10) Pub. No.: US 2025/0143612 A1
(43) Pub. Date: May 8, 2025

(54) INFORMATION PROCESSING DEVICE, INFORMATION PROCESSING METHOD, PROGRAM, AND INFORMATION PROCESSING SYSTEM

(71) Applicant: SONY GROUP CORPORATION, TOKYO (JP)

(72) Inventors: MAO KATSUHARA, TOKYO (JP); TAKANORI ISHIKAWA, TOKYO (JP); YUTA KONDO, TOKYO (JP); RIHO HITSUYU, TOKYO (JP)

(21) Appl. No.: 18/838,321

(22) PCT Filed: Feb. 6, 2023
(86) PCT No.: PCT/JP2023/003705

§ 371 Date: Aug. 14, 2024

(30) Foreign Application Priority Data
Feb. 22, 2022 (JP) 2022-025978

(51) Int. Cl. A61B 5/16; A61B 5/0245

(52) U.S. Cl. CPC A61B 5/165; A61B 5/0245

(57) ABSTRACT

The present technology relates to an information processing device, an information processing method, a program, and an information processing system that allow for improvement in the accuracy of emotion estimation using biological information. An information processing device according to an aspect of the present technology generates, on the basis of biological information indicating a biological reaction measured by a first device when a user is at rest, reference information indicating the biological reaction to be used as a reference in emotion estimation and estimates emotion when the user is active on the basis of biological information indicating the biological reaction measured by a second device when the user is active and the reference information. The present technology can be applied to emotion estimation using user's biological information.

### TECHNICAL FIELD

The present technology relates to an information processing device, an information processing method, a program, and an information processing system, and more particularly to an information processing device, an information processing method, a program, and an information processing system that allow for improvement in the accuracy of emotion estimation using biological information.

### BACKGROUND ART

In recent years, wearable devices in the form of earphones and headphones, known as “hearable devices,” have become widely available. Among such hearable devices, some equipped with vital sensors like heart rate sensors and EEG sensors have been proposed. Acquiring biological information inside and around the ears allows for managing the user's physical conditions, performing emotion estimation, and providing services based on the result of emotion estimation.

PTL 1 and PTL 2 disclose features for estimating physical conditions and emotions using vital sensors.

### CITATION LIST

### Patent Literature

### [PTL 1]

JP 2020-096679A

### [PTL 2]

JP 2018-018492A

### SUMMARY

### Technical Problem

A reference value is required in emotion estimation. In order to improve the accuracy of estimation, it is necessary to accurately acquire biological information, for example at rest, which serves as a reference. The same is true for emotion estimation based on biological information acquired in a hearable device.

In view of the foregoing, the present technology is directed to improvement in emotion estimation using biological information.

### Solution to Problem

An information processing device according to an aspect of the present technology includes a generation unit configured to generate, on the basis of biological information indicating a biological reaction measured by a first device when a user is at rest, reference information indicating the biological reaction to be used as a reference in emotion estimation, and a first emotion estimation unit configured to estimate emotion when the user is active on the basis of biological information indicating the biological reaction measured by a second device when the user is active and the reference information.

According to an aspect of the present technology, on the basis of biological information indicating a biological reaction measured by a first device when a user is at rest, reference information indicating the biological reaction to be used as a reference in emotion estimation is generated, and emotion when the user is active is estimated on the basis of biological information indicating the biological reaction measured by a second device when the user is active and the reference information.

### BRIEF DESCRIPTION OF DRAWINGS

FIG. 1 is a view of an exemplary information processing system according to an embodiment of the present technology.

FIG. 2 is a flowchart for illustrating the flow of processing in the information processing system in FIG. 1.

FIG. 3 is a block diagram of an exemplary hardware configuration of a smartphone.

FIG. 4 is a block diagram of an exemplary functional configuration of a controller.

FIG. 5 is a flowchart for illustrating processing by the smartphone.

FIG. 6 is a flowchart for illustrating emotion estimation processing performed in step S5 in FIG. 5.

FIG. 7 is a chart for illustrating an example of the operation concept of an information processing system.

FIG. 8 shows an example of transitions between sleep stages and heart rate variability.

FIG. 9 is a block diagram of an exemplary hardware configuration of a hearable device.

FIG. 10 is a view for illustrating another example of an auxiliary device.

### DESCRIPTION OF EMBODIMENTS

Hereinafter, modes for carrying out the present technology will be described.

The description will be made in the following order.
1. Outline of present technique
2. Configuration of information processing device
3. Operation of information processing device
4. Modifications

### 1. Outline of Present Technology

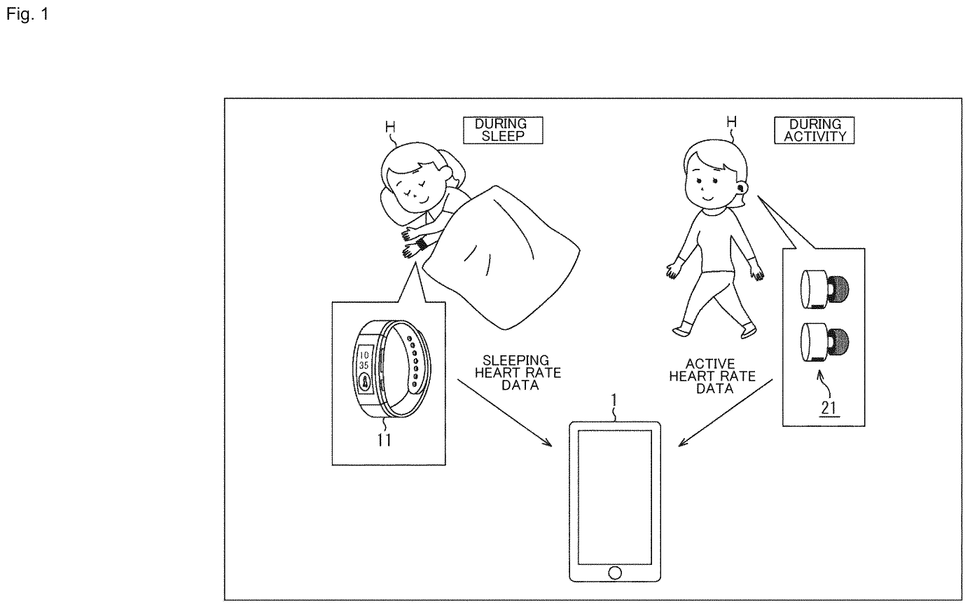

FIG. 1 is a view of an exemplary information processing system according to an embodiment of the present technology.

An information processing system according to an embodiment of the present technology includes a smartphone 1, an auxiliary device 11, and a hearable device 21. The auxiliary device 11 and the hearable device 21 are each connected to the smartphone 1 through wireless communication for example by Bluetooth (registered trademark) or wireless LAN technology.

The auxiliary device 11 and the hearable device 21 are used by the same person, user H. In the example in FIG. 1, the auxiliary device 11 is a wrist band type wearable device, for example, a so-called smartwatch. Meanwhile, the hearable device 21 includes completely standalone earphones worn on the ears. A headphone type hearable device may also be used as the hearable device 21.

The auxiliary device 11 and the hearable device 21 are equipped with a heart rate sensor that measures heart rate as a biological reaction. The heart rate sensor is a biosensor such as a PPG (Photo Plethysmography) sensor.

Any of various other sensors can be used as the heart rate sensor, such as a laser Doppler interferometer, a pressure sensor, a microphone (sound wave), an acceleration sensor, an image sensor (RGB or IR), a bolometer (thermo viewer), and a sensor using radio waves (radar) may be used instead of the PPG sensor.

As shown in the upper left part of FIG. 1, the auxiliary device 11 is worn around the wrist of the user H for example during sleep. Heart rate data during sleep, which is heart rate data measured by the auxiliary device 11 while the user H is asleep, is transmitted to the smartphone 1.

Meanwhile, as shown in the upper right part of FIG. 1, the hearable device 21 is worn on the ears of the user H for example during activity. Any time except for sleeping, such as the time the user H spends doing household chores at home or time spent outside, is activity time Active heart rate data, which is heart rate data measured by the hearable device 21 when the user H is active, is transmitted to the smartphone 1.

As will be described, in addition to the heart rate sensor, the hearable device 21 is also provided with an EEG sensor, which is a biosensor used to measure electroencephalogram (EEG) as another biological reaction. EEG data, which is brain wave data measured by the EEG sensor when the user H is active, is also transmitted to the smartphone 1.

The smartphone 1 estimates the emotion of the user H during activity, on the basis of heart rate data during sleep transmitted from the auxiliary device 11 and the active heart rate data transmitted from the hearable device 21.

Sleeping heart rate data represents the heart rate of the user H during sleep, in other words, at rest. In emotion estimation, biological information at rest is required as a reference, and sleeping heart rate data can be used as the reference information.

The emotion of the user H during activity is estimated by the smartphone 1 using, as the reference data (baseline data), information acquired on the basis of the sleeping heart rate data, which represents the resting heart rate. For example, wakefulness, which represents the consciousness level of the user H, is estimated as the emotion of the user H during activity.

The smartphone 1 also identifies a resting interval when the user H is active on the basis of a result of emotion estimation based on heart rate. The smartphone 1 estimates the emotion of the user H at each timing during activity on the basis of EEG data in the resting interval measured by the hearable device 21 and EEG data measured at each timing. For example, wakefulness, which represents the level of pleasantness/unpleasantness experienced by the user H, is estimated as the emotion of the user H during activity.

The smartphone 1 integrates the wakefulness estimated on the basis of heart rate and the wakefulness estimated on the basis of brain waves and provides various services to the active user H according to the integration result. Hereinafter, the wakefulness estimated on the basis of heart rate will be referred to as heart rate wakefulness, and the wakefulness estimated on the basis of brain waves will be referred to as brain wave wakefulness, as appropriate.

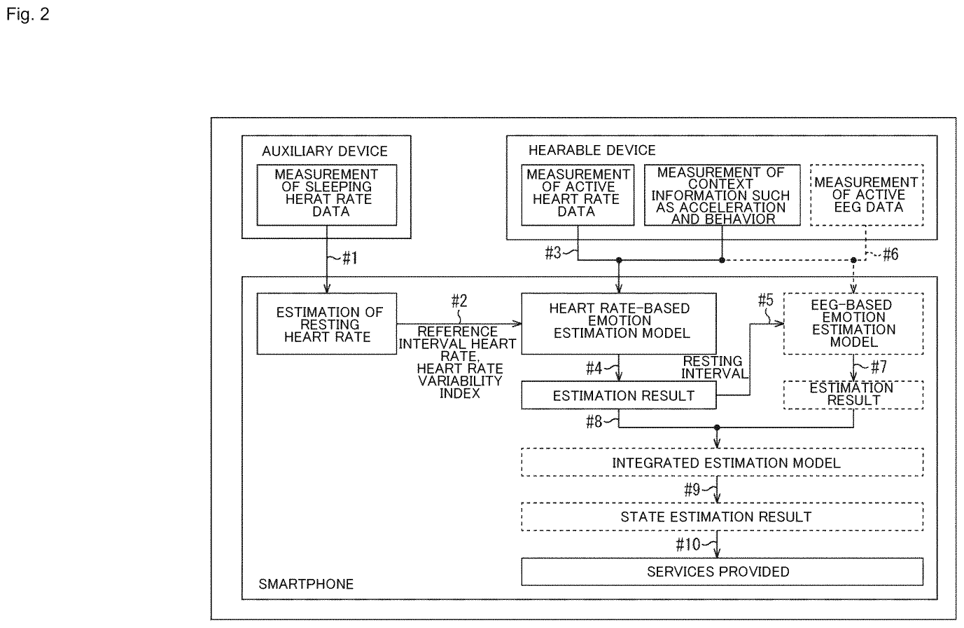

FIG. 2 is a flowchart for illustrating the flow of processing in the information processing system in FIG. 1

As shown in the upper left part of FIG. 2, the auxiliary device 11 measures sleeping heart rate. The sleeping heart rate data representing heart rate during sleep measured by the auxiliary device 11 is transmitted to the smartphone 1 as indicated by arrow #1.

The smartphone 1 estimates information about the resting heart rate on the basis of the sleeping heart rate data transmitted from the auxiliary device 11. For example, according to a prescribed algorithm, values for heart rate and heart rate variability index are estimated as information about the resting heart rate.

The heart rate variability index is expressed for example by at least any one of the following values.
HR (the average heart rate over a fixed period of time such as 5 minutes).
MeanPR (the average peak interval for heart rate over a fixed period of time such as 5 minutes).
SDNN (the standard deviation of the NN intervals over a fixed period of time such as 5 minutes).
RMSSD (the root mean square of successive differences between adjacent NN intervals)
NN50 (the number of pairs of successive NN intervals that differ by more than 50 ms).
pNN50 (the percentage of pairs of successive NN intervals that differ by more than 50 ms).
SD1 (the standard deviation on the ordinate in a scatter plot).
SD2 (the standard deviation on the abscissa in a scatter plot)

Intervals (periods) for estimating heart rate variability index include resting state intervals as follows.
An interval around a peak of heart rate variability which corresponds to shallow sleep.
An interval immediately before waking up
An interval immediately after waking up

The measurement time is desirably at least five minutes. By utilizing other kinds of information such as acceleration, the measurement time can be set to at least 30 seconds and less than 5 minutes.

The information about resting heart rate is input into a heart rate-based emotion estimation model as heart rate information in the reference interval as indicated by arrow #2. The heart rate-based emotion estimation model takes for example the heart rate information in the reference interval and active heart rate data as input and outputs emotion (“heart rate wakefulness”). An estimation model including for example a neural network generated in advance by machine learning is prepared in the smartphone 1.

Meanwhile, as shown in the upper center part of FIG. 2, active heart rate is measured at the hearable device 21. The active heart rate data, which represents the heart rate during activity measured at the hearable device 21, is transmitted to the smartphone 1 and input into the heart rate-based emotion estimation model, as indicated by arrow #3. Information about the acceleration and behavior of the user H during activity may be measured at the hearable device 21 as context information, and the context information may be used for the heart rate-based emotion estimation.

Emotion estimation is performed by the heart rate-based emotion estimation model, and the result of the emotion estimation, heart rate wakefulness, is output, as indicated by arrow #4.

The smartphone 1 identifies a resting interval during activity, on the basis of the result of emotion estimation by the heart rate-based emotion estimation model. For example, an interval, in which a resting state is expressed by heart rate wakefulness, is identified as the resting interval. The information about the resting interval is input into the EEG-based emotion estimation model, as indicated by arrow #5.

As shown in the upper right part of FIG. 2, brain waves during activity are measured at the hearable device 21. The active EEG data representing brain waves during activity measured at the hearable device 21 is transmitted to the smartphone 1 and input into the EEG-based emotion estimation model as indicated by arrow #6.

The EEG-based emotion estimation model for example takes information about the resting interval and the active EEG data as input and outputs emotion (EEG wakefulness). The estimation model generated in advance by machine learning and including for example a neural network is provided in the smartphone 1. Context information may be used for the EEG-based emotion estimation.

After emotion estimation is performed by the EEG-based emotion estimation model, the result of the emotion estimation, EEG wakefulness, is output as indicated by arrow #7.

Heart rate wakefulness, which is the result of the heart rate-based emotion estimation model, and EEG wakefulness, which is the result of the EEG-based emotion estimation model, are input to an integrated estimation model, as indicated by arrow #8.

The integrated estimation model for example uses heart rate wakefulness and EEG wakefulness as inputs and outputs the physical condition of the user H.

The state estimation is performed by the integrated estimation model, and the result of the state estimation is output, as indicated by arrow #9.

Various services are provided to the active user H on the basis of the result of the state estimation, as indicated by arrow #10. For example, a song is played according to the state of the user H, or a content other than music is provided.

Resting heart rate provides useful information for estimating physical conditions and emotions since disturbances are relatively minimal throughout the day.

However, it is difficult to measure heart rate during sleep (which is resting time) using the hearable device because it is often the case that the hearable device is removed during sleep.

In the information processing system in FIG. 1, the auxiliary device 11, which is a different modal from the hearable device 21, is used to measure sleeping heart rate. The use of the auxiliary device 11, which is a wristband-type device, for measuring heart rate during sleep allows the heart rate of the user H at rest to be measured more accurately than when the hearable device 21 is used during sleep.

In addition, using accurately measured resting heart rate as a reference for emotion estimation, the emotion estimation at each timing during activity can also be performed with high accuracy.

Furthermore, there are individual differences in biological reactions such as heart rate. The use of biological reactions at rest as a reference allows for emotion estimation that takes such individual differences into account and enables services to be optimized for the individuals on the basis of the result of emotion estimation that takes individual differences into account.

In this way, according to the information processing system in FIG. 1, heart rate measured during sleep by the auxiliary device 11 can be used as a reference for heart rate-based emotion estimation using the heart rate measured during activity by the hearable device 21, so that the accuracy of emotion estimation can be improved and services can be optimized for each individual.

A series of operations carried out by the smartphone 1 configured to perform emotion estimation as described above will be described later with reference to a flowchart.

### 2. Configuration of Information Processing Device

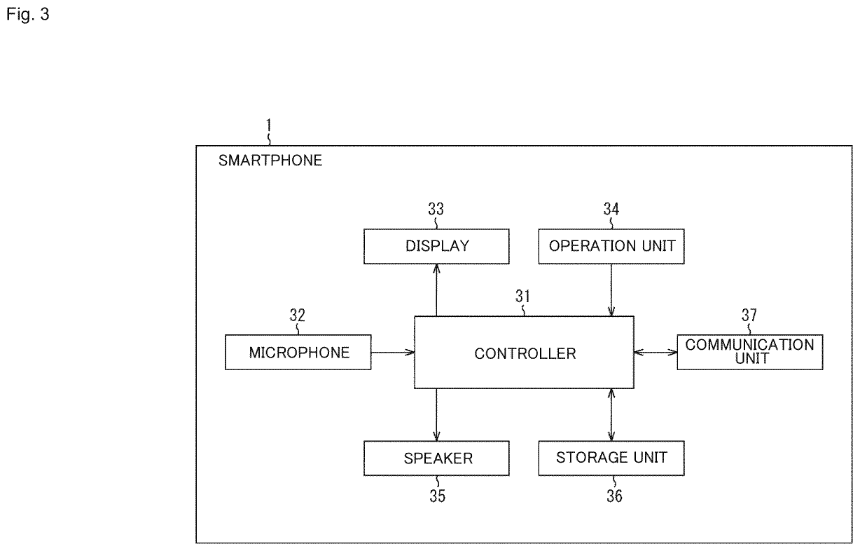

FIG. 3 is a block diagram of an exemplary hardware configuration of the smartphone 1.

The smartphone 1 includes a controller 31, and a microphone 32, a display 33, an operation unit 34, a speaker 35, a storage unit 36, and a communication unit 37 connected to the controller 31.

The controller 31 may include a CPU (Central Processing Unit), a ROM (Read Only Memory), and a RAM (Random Access Memory). The controller 31 executes a prescribed program to control the overall operation of the smartphone 1. For example, the controller 31 estimates the emotion of the user H on the basis of biological information transmitted from the auxiliary device 11 and the hearable device 21.

The microphone 32 outputs collected data, such as voice, to the controller 31.

The display 33 displays various kinds of information about the result of emotion estimation, such as a presentation screen, under the control of the controller 31.

The operation unit 34 includes operation buttons and a touch panel, and other elements provided on the surface of the casing of the smartphone 1. The operation unit 34 outputs information indicating the content of operation carried out by the user to the controller 31.

The speaker 35 outputs sound according to sound data supplied from the controller 31.

The storage unit 36 may include a flash memory or a memory card inserted into a card slot provided in the casing. The storage unit 36 stores various kinds of data supplied by the controller 31.

The communication unit 37 includes a communication module such as a wireless LAN or Bluetooth (registered trademark). The communication unit 37 communicates with an external device used by the user H and a server on the Internet. For example, the communication unit 37 receives various kinds of information such as biological information transmitted from the auxiliary device 11 and the hearable device 21 and outputs these kinds of information to the controller 31.

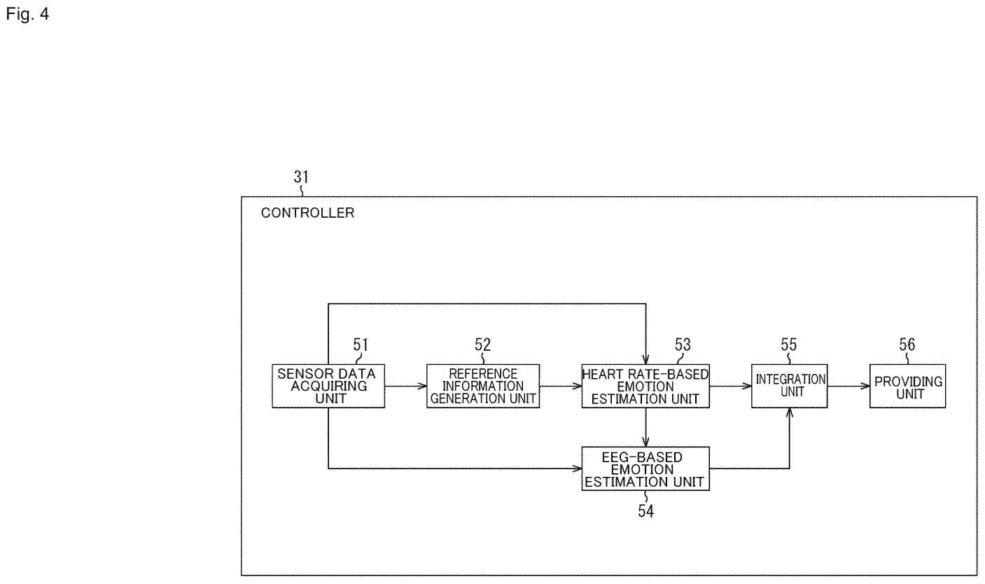

FIG. 4 is a block diagram of an exemplary functional configuration of the controller 31.

The controller 31 includes a sensor data acquiring unit 51, a reference information generation unit 52, a heart rate-based emotion estimation unit 53, an EEG-based emotion estimation unit 54, an integration unit 55, and a providing unit 56. At least some of the functional units shown in FIG. 4 are implemented by causing the CPU that constitutes the controller 31 to execute a prescribed program.

The sensor data acquiring unit 51 acquires sleeping heart rate data transmitted from the auxiliary device 11 and received at the communication unit 37. The sensor data acquiring unit 51 acquires the active heart rate data, the active EEG data, and context information transmitted from the hearable device 21 and received at the communication unit 37.

The sleeping heart rate data acquired by the sensor data acquiring unit 51 is output to the reference information generation unit 52, and active heart rate data and context information are output to the heart rate-based emotion estimation unit 53. The active EEG data and the context information acquired by the sensor data acquiring unit 51 are output to the EEG-based emotion estimation unit 54.

The reference information generation unit 52 generates reference information indicating a biological reaction to be used as a reference in emotion estimation on the basis of the sleeping heart rate data supplied from the sensor data acquiring unit 51. The reference information generation unit 52 generates, for example, values for heart rate and heart rate variability index at rest as the reference information. The information generated by the reference information generation unit 52 is output to the heart rate-based emotion estimation unit 53.

The heart rate-based emotion estimation unit 53 estimates the emotion of the user H during activity on the basis of the heart rate information in the reference interval indicated by the reference information supplied by the reference information generation unit 52 and the active heart rate data supplied by the sensor data acquiring unit 51. For example, the heart rate and the heart rate variability index during activity are generated on the basis of the active heart rate data and are used for emotion estimation together with the heart rate information in the reference interval. The information on heart rate wakefulness, which is the result of the estimation by the heart rate-based emotion estimation unit 53, is output to the integration unit 55.

The heart rate-based emotion estimation unit 53 identifies a resting interval when the user H is active on the basis of the result of the emotion estimation. The information about the resting interval identified by the heart rate-based emotion estimation unit 53 is output to the EEG-based emotion estimation unit 54.

The EEG-based emotion estimation unit 54 estimates the emotion of the user H during activity on the basis of the information on the resting interval supplied by the heart rate-based emotion estimation unit 53 and the active EEG data supplied by the sensor data acquiring unit 51. The information on EEG wakefulness, which is the result of the estimation by the EEG-based emotion estimation unit 54, is output to the integration unit 55.

The integration unit 55 integrates the results of the emotion estimation by the heart rate-based emotion estimation unit 53 and the EEG-based emotion estimation unit 54 to estimate the state of the user H. The information indicating the state of the user H estimated by integration unit 55 is output to the providing unit 56.

The providing unit 56 provides various services to the user H during activity on the basis of the information supplied by the integration unit 55. For example, music is played according to the state of the user H, and the playback sound is output from the speaker 35 or the hearable device 21. Also, a video is played according to the state of user H, and the image is displayed on display 33. The results of the heart rate-based emotion estimation, such as time series data on heart rate wakefulness, time series data on EEG wakefulness, and time series data on the integration result of the heart rate wakefulness and the EEG wakefulness, may be presented by the providing unit 56.

### 3. Operation of Information Processing Device

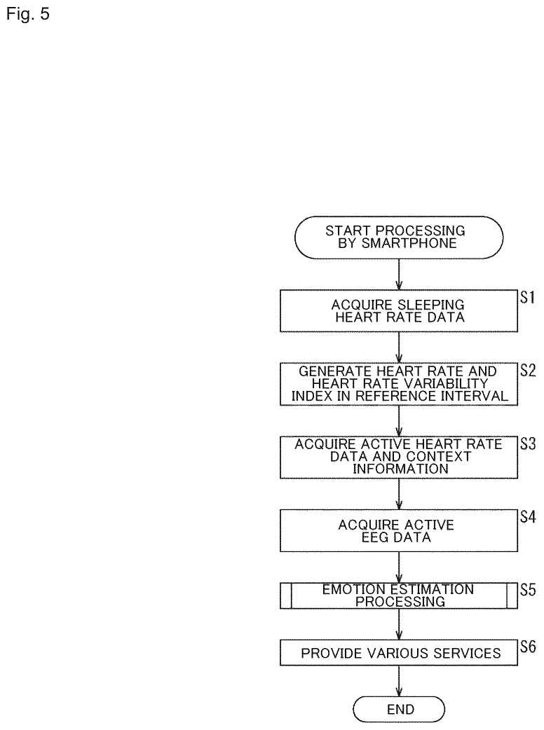

With reference to the flowchart in FIG. 5, the processing performed by the smartphone 1 will be described. The processing in FIG. 5 starts for example as sleeping heart rate data measured by the auxiliary device 11 is transmitted.

In step S1, the sensor data acquiring unit 51 acquires sleeping heart rate data transmitted from the auxiliary device 11.

In step S2, the reference information generation unit 52 generates reference information indicating heart rate and heart rate variability index in a reference interval on the basis of the sleeping heart rate data.

In step S3, the sensor data acquiring unit 51 acquires active heart rate data and context information transmitted from the hearable device 21.

In step S4, the sensor data acquiring unit 51 acquires active EEG data transmitted from the hearable device 21.

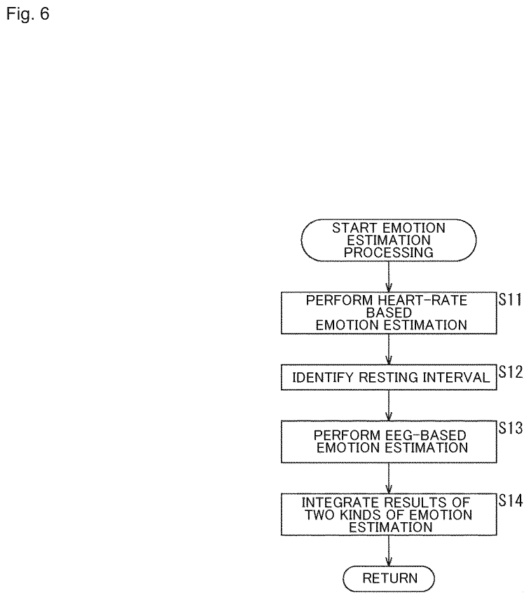

In step S5, emotion estimation processing is performed. The emotion estimation processing will be described later with reference to the flowchart in FIG. 6.

In step S6, the providing unit 56 provides various services to the user H on the basis of the result of the emotion estimation.

Next, with reference to the flowchart in FIG. 6, the emotion estimation processing performed in step S5 in FIG. 5 will be described.

In step S11, the heart rate-based emotion estimation unit 53 performs heart rate-based emotion estimation on the basis of the heart rate information in the reference interval indicated by the reference information and the active heart rate data.

In step S12, the heart rate-based emotion estimation unit 53 identifies a resting interval during activity on the basis of the result of the heart rate-based emotion estimation. For example, labeling is performed to indicate a resting interval in time-series data as the result of the emotion estimation for the user H.

In step S13, the EEG-based emotion estimation unit 54 performs EEG-based emotion estimation on the basis of active EEG data. The EEG-based emotion estimation is performed on the basis of brain waves in the resting interval as a reference.

In step S14, the integration unit 55 integrates the results of the heart rate-based emotion estimation and the EEG-based emotion estimation. Thereafter, the process returns to step S5 in FIG. 5, and further processing is performed.

Through the series of processing steps, the smartphone 1 uses biological information measured by the auxiliary device 11 as biological information in the reference interval, thereby improving the accuracy of emotion estimation. In addition, the smartphone 1 can provide services optimized for each individual on the basis of the result of emotion estimation that takes individual differences into account.

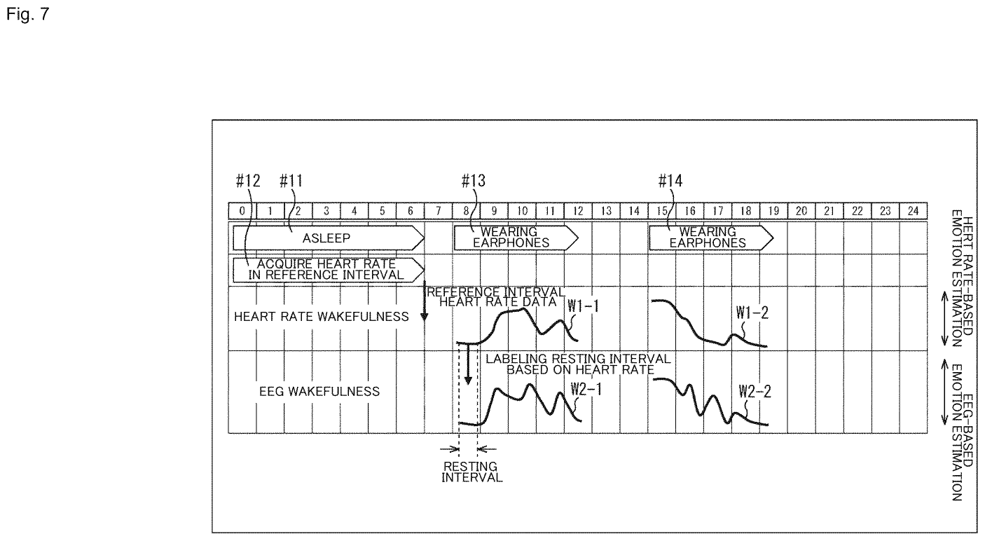

FIG. 7 is a chart for illustrating an example of the operation concept of the information processing system.

The time axis in FIG. 7 represents hourly segments from 0:00 to 24:00. In the example in FIG. 7, the user H is asleep from 0:00 to 7:00, as indicated by arrow #11. The heart rate in the time period is measured by the auxiliary device 11, and information on the heart rate in the reference interval is acquired, as indicated by arrow #12.

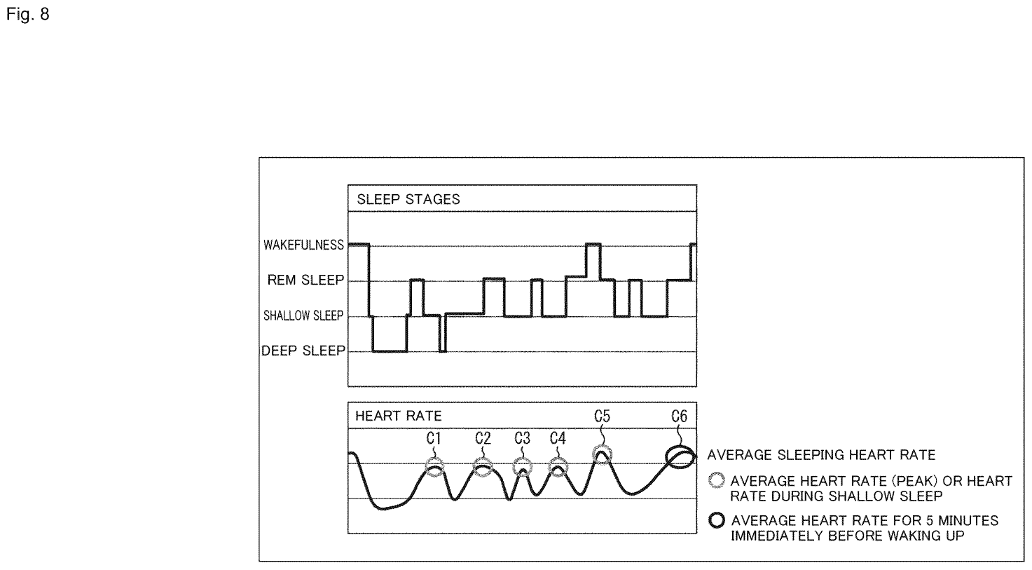

FIG. 8 shows an example of transitions between sleep stages and heart rate variability. The abscissa in FIG. 8 represents time.

As shown in the upper part of FIG. 8, the sleep stage is represented by four stages, i.e., wakefulness, REM sleep, shallow sleep, and deep sleep. For example, the sleep stage is estimated on the basis of information from an acceleration sensor and heart rate information.

As shown in the lower part of FIG. 8, the heart rate changes with the sleep stage. Basically, as the sleep stage approaches wakefulness, the heart rate value increases, and as the sleep stage approaches deep sleep, the heart rate value decreases.

For example, on the basis of heart rate information in the intervals indicated by circles C1 to C6, heart rate information in a reference interval to be reference information is acquired. For example, as indicated by circles C1 to C5, the average of the peak values for heart rate or heart rate in an interval of shallow sleep is acquired as heart rate information in the reference interval. An average value for heart rate during five minutes immediately before waking up, as indicated by circle C6, is acquired as heart rate information in the reference interval.

In this way, the reference information indicating a biological reaction to be used as a reference in emotion estimation is generated using a single value or an average for heart rate and heart rate variability in a peak interval of heart rate or in a prescribed interval.

Referring back to FIG. 7, the user H wears the hearable device 21 (earphones) between 8:00 and 12:30, as indicated by arrow #13. The user H's heart rate and brain waves in this time period are measured by the hearable device 21.

In the smartphone 1, as shown in the middle part of FIG. 7, heart rate-based emotion estimation is performed on the basis of heart rate information in a reference interval acquired on the basis of heart rate measured during sleep and activity heart rate data measured at each timing from 8:00 to 12:30. Changes in heart rate wakefulness at each timing are acquired by the heart rate-based emotion estimation. The waveform W1-1 in FIG. 7 shows changes in heart rate wakefulness.

In the smartphone 1, a resting interval is identified (labeled) on the basis of the result of heart rate-based emotion estimation. In the example in FIG. 7, for example, the interval indicated by the dashed line with a small change in heart rate wakefulness is identified as the resting interval.

In the smartphone 1, as shown in the lower part of FIG. 7, EEG-based emotion estimation is performed on the basis of EEG information in a resting interval and active EEG data measured at each timing from 8:00 to 12:30. Through the EEG-based emotion estimation, changes in EEG wakefulness at each timing are acquired. The waveform W2-1 in FIG. 7 indicates changes in EEG wakefulness.

The same processing as above is performed when the user H wears the hearable device 21. For example, when the user H wears the hearable device 21 from 15:00 to 19:30, as indicated by arrow #14, heart rate wakefulness and EEG wakefulness in this time period are acquired. The waveform W1-2 in FIG. 7 indicates changes in heart rate wakefulness during the time period from 15:00 to 19:30, and the waveform W2-2 indicates changes in EEG wakefulness during the time period from 15:00 to 19:30.

### 4. Modifications

### Example of Emotion Estimation Performed in Hearable Device 21

In the above description, emotion estimation is performed in the smartphone 1, but the estimation may also be performed in the hearable device 21.

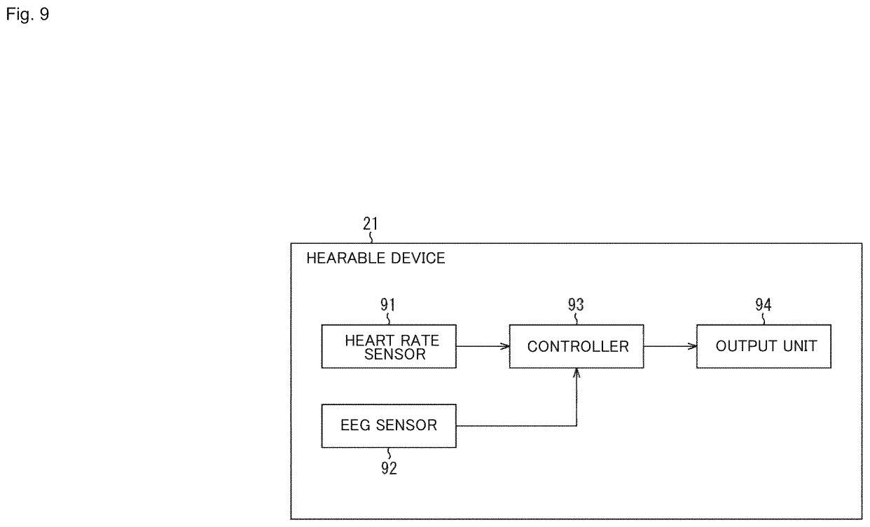

FIG. 9 is a block diagram of an exemplary hardware configuration of the hearable device 21 when emotion estimation is performed in the hearable device 21.

The hearable device 21 includes a heart rate sensor 91, an EEG sensor 92, a controller 93, and an output unit 94.

The heart rate sensor 91 measures the heart rate of the user H during activity. The active heart rate data is output to the controller 93.

The EEG sensor 92 measures the brain waves of the user H during activity. The active EEG data is output to the controller 93.

When emotion estimation is performed in the hearable device 21, each of the functions in FIG. 4 is implemented by the controller 93. The controller 93 estimates the emotion of the user H on the basis of biological information supplied by the heart rate sensor 91 and the EEG sensor 92 and biological information transmitted from the auxiliary device 11 in the above-described manner. The controller 93 outputs the result of the emotion estimation to the output unit 94.

The output unit 94 plays music, etc., that matches the state of the user H according to the result of the emotion estimation supplied from the controller 93.

In this way, the function of emotion estimation can be provided in the 21 hearable devices.

At least any one of the heart rate-based emotion estimation and the EEG-based emotion estimation may be performed on a server on the Internet.

In the above description, the result of heart rate-based emotion estimation is extended to EEG-based emotion estimation, while the result may be extended to emotion estimation based on biological information indicating any of other biological reactions, such as emotion estimation based on electromyography/electrocardiography or psychogenic perspiration. In this case, the hearable device 21 is provided with a biopotential sensor for measuring electromyography and electrocardiography and an impedance sensor for measuring psychogenic perspiration.

### Other Examples of Auxiliary Device 11

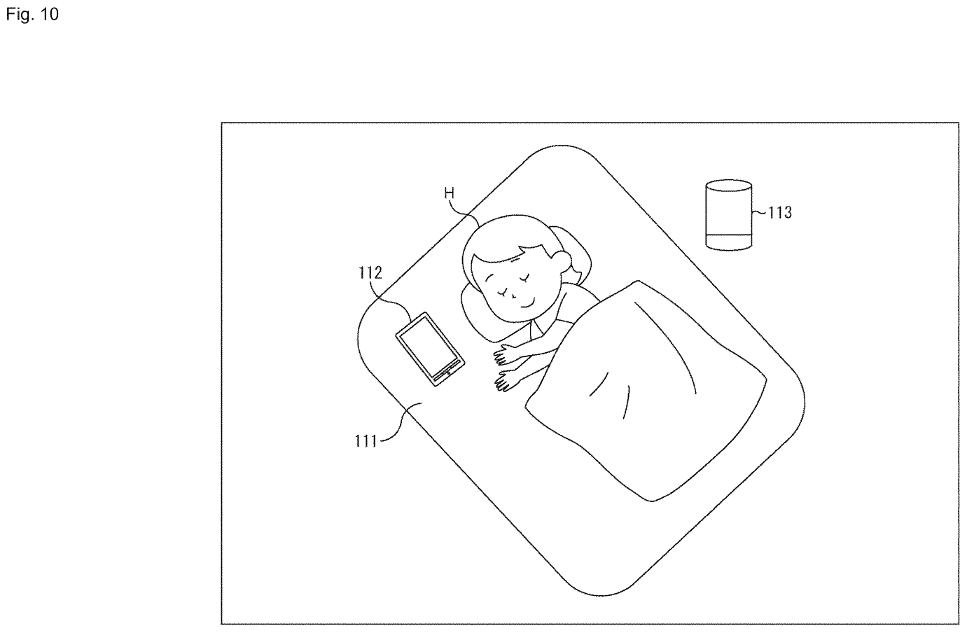

FIG. 10 is a view for illustrating another example of the auxiliary device 11.

In the above description, a wristband type wearable device such as a smartwatch is used as the auxiliary device 11, while a mobile device such as a smartphone, any of other wearable devices such as a health care monitor, and a stationary device such as a smart speaker and smart bedding may also be used as the auxiliary device 11. The smart bedding for example refers to bedding such as a mattress equipped with a sensor for measuring a biological reaction and a communication module for communication with the smartphone 1.

In the example in FIG. 10, smart bedding 111, a smartphone 112 which is a different device from the smartphone 1, and a smart speaker 113 are provided near the user H. The sleeping heart rate of the user H is measured by any of these devices and the sleeping heart rate data is transmitted to the smartphone 1.

The device for measuring the heart rate of the user H during sleep may or may not make contact with the user H. In this way, various devices that do not disturb the sleep of the user H can be used as devices to measure the sleeping heart rate.

### Program

The above-described series of processing steps can be executed by hardware or software. When executing the series of processing steps by software, a program that makes up the software is installed from a program recording medium onto either a computer incorporated in dedicated hardware or a general-purpose personal computer.

The program to be installed is recorded and provided for example on a removable medium or provided via a wired or wireless transmission medium such as a local area network, the Internet, and digital broadcasting.

The program executed by the computer may proceed chronologically according to the sequence described herein or may be executed in parallel or at necessary timings such as when called.

Herein, the term “system” refers to a collection of multiple elements (devices, modules (parts), etc.), and all the elements may or may not be located within the same casing. Therefore, multiple devices stored in separate casings and connected over a network and a single device having a plurality of modules stored in a single casing are both systems.

The advantageous effects described herein are merely examples and are not intended as limiting, and other advantageous effects may be acquired.

The embodiments of the present technology are not limited to the aforementioned embodiments, and various changes can be made without departing from the gist of the present technology.

For example, the present technology may have a cloud computing configuration in which a single function is shared among multiple devices over a network for cooperative processing.

In addition, each step described in the above flowchart can be executed by a single device or executed in a shared manner by a plurality of devices.

Furthermore, if a single step includes multiple kinds of processing, these multiple kinds of processing included in the single step can be executed by one device or shared and executed by multiple devices.

### Combination Examples of Configurations

The present technology can be configured as follows.

(1)

An information processing device comprising:
a generation unit configured to generate, on the basis of biological information indicating a biological reaction measured by a first device when a user is at rest, reference information indicating the biological reaction to be used as a reference in emotion estimation; and
a first emotion estimation unit configured to estimate emotion when the user is active on the basis of biological information indicating the biological reaction measured by a second device when the user is active and the reference information.
   (2)

The information processing device according to (1), wherein the generation unit generates the reference information using biological information indicating the biological reaction measured by the first device during sleep as biological information when the user is at rest.

(3)

The information processing device according to (1) or (2), wherein the biological reaction measured by the first and second devices is heart rate.

(4)

The information processing device according to (3), wherein the generation unit generates information to be used as a reference for each of heart rate and heart rate variability as the reference information, and
the first emotion estimation unit estimates emotion on the basis of biological information indicating heart rate and heart rate variability during activity and the reference information.
   (5)

The information processing device according to (3) or (4), wherein the generation unit generates the reference information indicating a single value or an average value for both heart rate and heart rate variability in a peak interval of heart rate.

(6)

The information processing device according to any one of (3) to (5), wherein the generation unit generates the reference information using biological information indicating heart rate and heart rate variability measured during a prescribed time period immediately before waking up as biological information when the user is at rest.

(7)

The information processing device according to any one of (3) to (6), wherein the generation unit generates the reference information using biological information indicating heart rate and heart rate variability measured during a prescribed time period immediately after waking up as biological information when the user is at rest.

(8)

The information processing device according to any one of (3) to (7), wherein a heart rate sensor provided in each of the first and second devices is at least any one of a photoplethysmographic sensor, a laser Doppler interferometer, a pressure sensor, a microphone, an acceleration sensor, an image sensor, a bolometer, and a sensor using radio waves.

(9)

The information processing device according to any one of (1) to (8), further comprising a second emotion estimation unit configured to estimate emotion when the user is active on the basis of biological information indicating another biological reaction measured by the second device in a resting interval in which the user is active, the resting interval being identified on the basis of a result of emotion estimation performed by the first emotion estimation unit and biological information indicating the other biological reaction when the user is active.

(10)

The information processing device according to (9), wherein the other biological reaction measured by the second device is brain waves.

(11)

The information processing device according to any one of (1) to (10), wherein the second device is a hearable device, and
the first device is a device different from the hearable device.
   (12)

The information processing device according to (11), wherein the first device is at least any one of a mobile device, a wearable device, and a stationary device.

(13)

An information processing method using an information processing device, wherein
the information device is configured to generate, on the basis of biological information indicating a biological reaction measured by a first device when a user is at rest, reference information indicating the biological reaction to be used as a reference in emotion estimation, and
estimate emotion when the user is active on the basis of biological information indicating the biological reaction measured by a second device when the user is active and the reference information.
   (14)

A program for causing a computer
to generate, on the basis of biological information indicating a biological reaction measured by a first device when a user is at rest, reference information indicating the biological reaction to be used as a reference in emotion estimation and
to estimate emotion when the user is active on the basis of biological information indicating the biological reaction measured by a second device when the user is active and the reference information.
   (15)

An information processing system comprising an information processing device, the information processing device comprising: a first device configured to measure a biological reaction when a user is at rest;
a second device configured to measure the biological reaction when the user is active;
a generation unit configured to generate, on the basis of biological information indicating the biological reaction measured by the first device when the user is at rest, reference information indicating the biological reaction to be used as a reference in emotion estimation; and
a first emotion estimation unit configured to estimate emotion when the user is active on the basis of biological information indicating the biological reaction measured by the second device when the user is active and the reference information.

### REFERENCE SIGNS LIST

1 Smartphone
11 Auxiliary device
21 Hearable device
51 Sensor data acquiring unit
52 Reference information generation unit
53 Heart rate-based emotion estimation unit
54 EEG-based emotion estimation unit
55 Integration unit
56 Providing unit

1. An information processing device comprising:
a generation unit configured to generate, on the basis of biological information indicating a biological reaction measured by a first device when a user is at rest, reference information indicating the biological reaction to be used as a reference in emotion estimation; and
a first emotion estimation unit configured to estimate emotion when the user is active on the basis of biological information indicating the biological reaction measured by a second device when the user is active and the reference information.

2. The information processing device according to claim 1, wherein the generation unit generates the reference information using biological information indicating the biological reaction measured by the first device during sleep as biological information when the user is at rest.

3. The information processing device according to claim 1, wherein the biological reaction measured by the first and second devices is heart rate.

4. The information processing device according to claim 3, wherein the generation unit generates information to be used as a reference for each of heart rate and heart rate variability as the reference information, and
the first emotion estimation unit estimates emotion on the basis of biological information indicating heart rate and heart rate variability during activity and the reference information.

5. The information processing device according to claim 3, wherein the generation unit generates the reference information indicating a single value or an average value for both heart rate and heart rate variability in a peak interval of heart rate.

6. The information processing device according to claim 3, wherein the generation unit generates the reference information using biological information indicating heart rate and heart rate variability measured during a prescribed time period immediately before waking up as biological information when the user is at rest.

7. The information processing device according to claim 3, wherein the generation unit generates the reference information using biological information indicating heart rate and heart rate variability measured during a prescribed time period immediately after waking up as biological information when the user is at rest.

8. The information processing device according to claim 3, wherein a heart rate sensor provided in each of the first and second devices is at least any one of a photoplethysmographic sensor, a laser Doppler interferometer, a pressure sensor, a microphone, an acceleration sensor, an image sensor, a bolometer, and a sensor using radio waves.

9. The information processing device according to claim 1, further comprising a second emotion estimation unit configured to estimate emotion when the user is active on the basis of biological information indicating another biological reaction measured by the second device in a resting interval in which the user is active, the resting interval being identified on the basis of a result of emotion estimation performed by the first emotion estimation unit and biological information indicating the other biological reaction when the user is active.

10. The information processing device according to claim 9, wherein the other biological reaction measured by the second device is brain waves.

11. The information processing device according to claim 1, wherein the second device is a hearable device, and
the first device is a device different from the hearable device.

12. The information processing device according to claim 11, wherein the first device is at least any one of a mobile device, a wearable device, and a stationary device.

13. An information processing method using an information processing device, wherein
the information device is configured to generate, on the basis of biological information indicating a biological reaction measured by a first device when a user is at rest, reference information indicating the biological reaction to be used as a reference in emotion estimation, and
estimate emotion when the user is active on the basis of biological information indicating the biological reaction measured by a second device when the user is active and the reference information.

14. A program for causing a computer
to generate, on the basis of biological information indicating a biological reaction measured by a first device when a user is at rest, reference information indicating the biological reaction to be used as a reference in emotion estimation and
to estimate emotion when the user is active on the basis of biological information indicating the biological reaction measured by a second device when the user is active and the reference information.

15. An information processing system comprising an information processing device, the information processing device comprising: a first device configured to measure a biological reaction when a user is at rest;
a second device configured to measure the biological reaction when the user is active;
a generation unit configured to generate, on the basis of biological information indicating the biological reaction measured by the first device when the user is at rest, reference information indicating the biological reaction to be used as a reference in emotion estimation; and
a first emotion estimation unit configured to estimate emotion when the user is active on the basis of biological information indicating the biological reaction measured by the second device when the user is active and the reference information.
